(19) United States
(12) Patent Application Publication
(10) Pub. No.: US 2012/0116259 A1
(43) Pub. Date: May 10, 2012

(54) BELT FOR TRAINING ABDOMINAL MUSCLES AND TRAINING METHOD EMPLOYING THE SAME

(76) Inventors: Alison Kay McConnell, Bournemouth (GB); Owen James Warren Evans, Hove (GB); Eden Richard Smith, London (GB); Jon Trevor Moakes, London (GB)

(21) Appl. No.: 13/261,043

(22) PCT Filed: May 26, 2010
(86) PCT No.: PCT/EP2010/057234

§ 371 Date: Jan. 20, 2012

(30) Foreign Application Priority Data
May 26, 2009 (GB) 0908906.1

(51) Int. Cl. A61B 5/11

(52) U.S. Cl. 600/595

(57) ABSTRACT

A belt for training abdominal muscles comprises means (103) for determining a base girth of a user. Means (115, 117, 119) is also provided for determining changes in girth of the user as a result of contraction and relaxation of the user's abdominal muscles. Further means (125) provides feedback to the user as to the extent of contraction of the user's abdominal muscles, the feedback being displayed as a continuous, progressive indication of the degree of contraction of the user's abdominal muscles. A training method employs the belt and comprises the steps of: placing the belt around the waist of a user and determining a base girth of the user. The user's abdominal muscles are contracted and relaxed so as to provide feedback to the user as to the extent of contraction of the user's abdominal muscles, and a continuous, progressive indication of the degree of contraction of the user's abdominal muscles is noted.

This invention relates to a belt for training abdominal muscles and training method employing the same.

Four groups of muscles enclose the body's abdominal compartment forming a box-like structure. These are: the diaphragm (top), transversus abdominis (back and sides), rectus abdominis (front), and pelvic floor muscles (floor) and are herein referred to collectively as “abdominal muscles”.

Simultaneous contraction of the abdominal muscles compresses the abdominal compartment, reducing its volume and raising its internal pressure. This rise in pressure and the traction generated by the muscles at their insertions to the bony structures of the body act to stabilise the spine and associated bony structures. The abdominal muscles are therefore useful in minimising the risk of injury and ensuring the effective transmission of force through the body. Training of the abdominal muscles is therefore desirable for injury prevention and rehabilitation, as well as being of concern for aesthetic reasons. Accordingly, the abdominal muscles are the focus of specific training by therapists, athletes and the general public.

The intra-abdominal pressure generated during contraction of the abdominal muscles opposes the contraction of these muscles. The intra-abdominal pressure therefore provides a resistance that can be harnessed and used as a training stimulus to the abdominal muscles. Without compression of the abdominal compartment the muscles receive no training stimulus and will not become stronger. That is, simply activating the muscles does not serve to improve their function: the muscles must shorten and compress the abdominal compartment in order to become stronger. The greater the contraction force, the greater the degree of abdominal compression, the greater the intra-abdominal pressure, and the greater the corresponding training stimulus to the abdominal muscles. Further, the ability to compress the abdominal compartment is proportional to the abdominal muscle strength. As strength increases, the ability to compress the abdominal compartment increases, which generates a proportionate increase in the training stimulus to the muscles; that is, there is automatic training progression.

There is, therefore, a need for a means, such as a belt, for determining the magnitude of abdominal compression so as to indicate the magnitude of the training stimulus to the abdominal muscles and for monitoring the progress of training.

The transversus abdominis muscles are vital abdominal compression muscles that are not activated in any meaningful way during conventional abdominal muscle training. Abnormal function of transversus abdominis muscles has been linked to low back pain. Further, lack of activity (detraining) of these vital abdominal compression muscles leads the abdominal wall to distend under the action of gravity. Conversely, specific training of transversus abdominis muscles leads to relief of low back pain and restraint of the abdominal contents, preventing the abdomen from distending.

The use of exercise belts is well known, for example from U.S. Pat. No. 5,857,984 which provides both a visual and a tactile alarm when the user's waist expands beyond a predetermined width. A disadvantage of such belts is that they only provide an alarm when the user's waist expands and not when the waist contracts and therefore respond to relaxation of the abdominal muscles rather than contraction. Thus, they do not provide an adequate means of improving the strength of the abdominal muscles. A further disadvantage is that such belts are therefore unable to monitor the progress of any training with time, and cannot be used as a means of harnessing the internal resistance of the abdominal pressure as a means of strengthening the abdominal muscles.

Additionally, previous inventions have required the user to maintain a continuous contraction of the muscles throughout the day, which means that the magnitude of the muscle contraction force is small (otherwise it could not be sustained), which means that the exercise is far less effective than when periodic bouts of “purposeful” strong muscle contractions are undertaken.

Prior art devices, such as that described in WO 2009/013490, do not permit the user, or a personal trainer/healthcare professional, to evaluate the effectiveness of the contraction, because they provide no immediate feedback of the extent of the change in waist girth that has been achieved during an attempted contraction of the transversus abdominis muscles. It has been found that this omission has the disadvantage that it prevents the user using the device to learn how to contract his transversus abdominis muscles to compress the abdominal compartment, thereby achieving an adequate training stimulus. The inability of many people to contract the transversus abdominis muscles is well known and therefore the provision of a device for accomplishing this is desirable.

It is therefore an object of the present invention to provide a belt for training abdominal muscles which overcomes or at least ameliorates the disadvantages of known exercise belts and to provide a training method employing such a belt.

According to one aspect of the present invention there is provided a belt for training abdominal muscles comprising: means for determining a base girth of a user; means for determining changes in girth of the user as a result of contraction and relaxation of the user's abdominal muscles; means for providing feedback to the user as to the extent of contraction of the user's abdominal muscles, the feedback means comprising means for displaying a continuous, progressive indication of the degree of contraction of the user's abdominal muscles.

The feedback means may include a scale which indicates the degree of contraction of the user's abdominal muscles.

The feedback means may include means, such as a scale, for indicating a maximal contraction of the user's abdominal muscles.

A belt member for extending around a waist of a user may be substantially inelastic. The belt member may be connected at each end thereof to the means for determining changes in the girth of the user, which means for determining changes includes means for adjusting the length of the belt member at each end thereof. The means for adjusting the length of the belt member may include a rotatable member. The means for adjusting the length of the belt member may include biasing means which tends to tighten the belt member around a user's waist. The means for adjusting the length of the belt member may comprise a rack and pinion assembly, a rack portion being attached to each end of the belt member and a rotatable pinion arranged in engagement with the two rack portions. Alternatively, the means for adjusting the length of the belt member may comprise a spool upon which two strips of the belt member are wound.

The belt may include means for determining when the girth of the user attains a predetermined threshold relative to the difference between the base girth and a girth corresponding to the user's maximum contraction of the abdominal muscles. The belt may include means for varying the feedback to the user as the user's girth varies beyond the predetermined threshold.

The means for providing feedback may include one or more of audio feedback means, visual feedback means, and tactile feedback means.

The belt may include means for recording the user's minimum girth and/or the user's base girth. The belt may include means for recording one or more of the girths over time.

The belt may include means for recording the duration of training and/or the duration and number of reductions in girth.

The belt may include means for providing the user with feedback when the user maintains the predetermined threshold for a predetermined time and/or when the user attains the predetermined threshold a predetermined number of times.

The belt may include a means of reminding the user that it is time to take a scheduled training session.

According to another aspect of the present invention there is provided a training method employing a belt as hereinbefore defined, the method comprising the steps of: placing the belt around the waist of a user and determining a base girth of the user; and contracting and relaxing the user's abdominal muscles so as to provide feedback to the user as to the extent of contraction of the user's abdominal muscles; and noting a continuous, progressive indication of the degree of contraction of the user's abdominal muscles.

The method may include the further step of the user inhaling while maintaining a reduction in abdominal girth.

The method may include the step of testing the endurance of the user's abdominal muscles. In such an endurance testing step the user may make repeated maximal contractions of the abdominal muscles in time with signals provided by the belt.

The method may include the step of deriving an index of abdominal muscle endurance by analysing the decline in girth reduction with time.

For a better understanding of the present invention and to show more clearly how it may be carried into effect reference will now be made, by way of example, to the accompanying drawings in which:

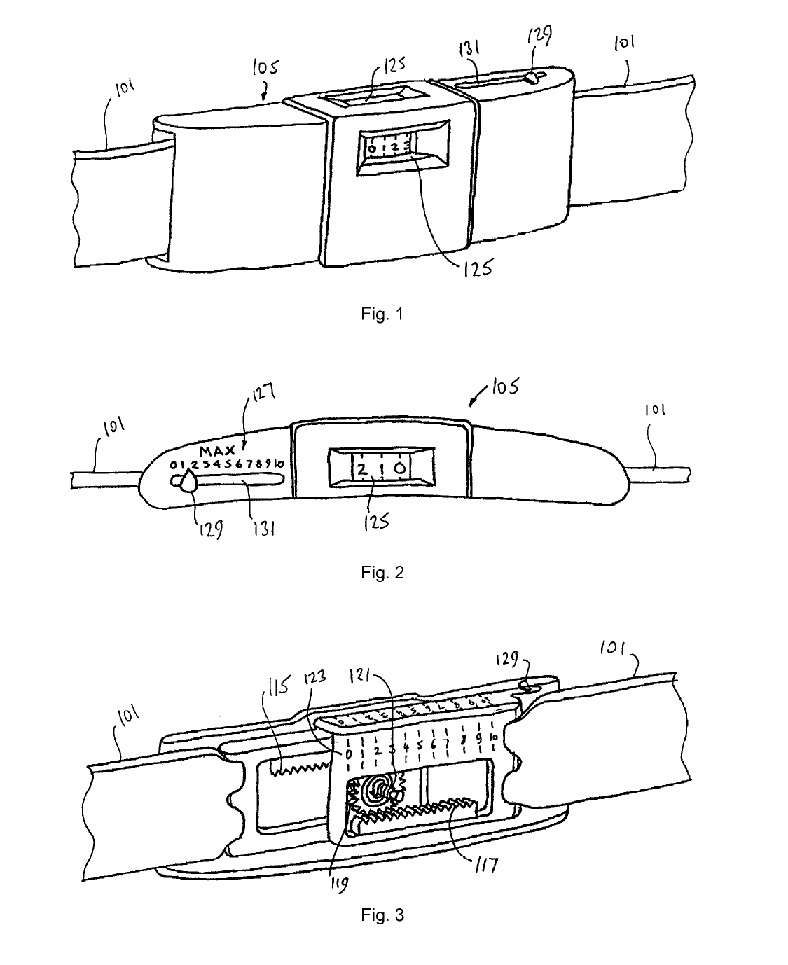

FIG. 1 is a front perspective view of part of one embodiment of a belt according to the present invention for training abdominal muscles;

FIG. 2 is a top view of the part of the belt shown in FIG. 1;

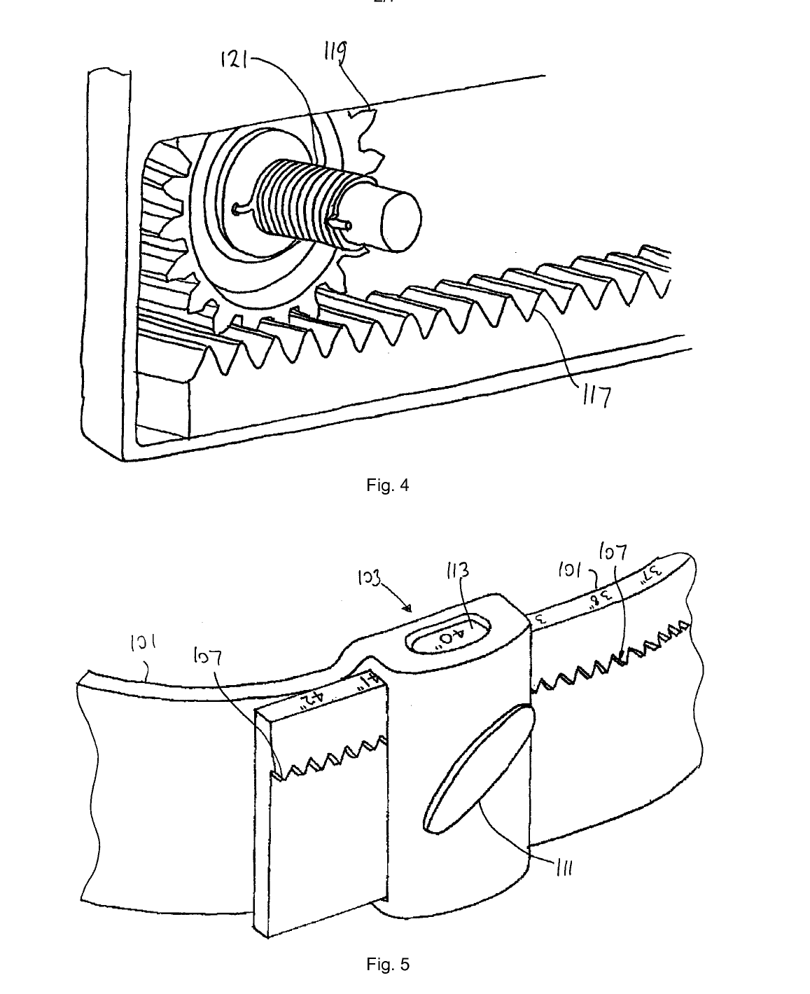

FIG. 3 is a front perspective view corresponding to FIG. 1 with a front cover removed;

FIG. 4 shows part of FIG. 3 on a larger scale;

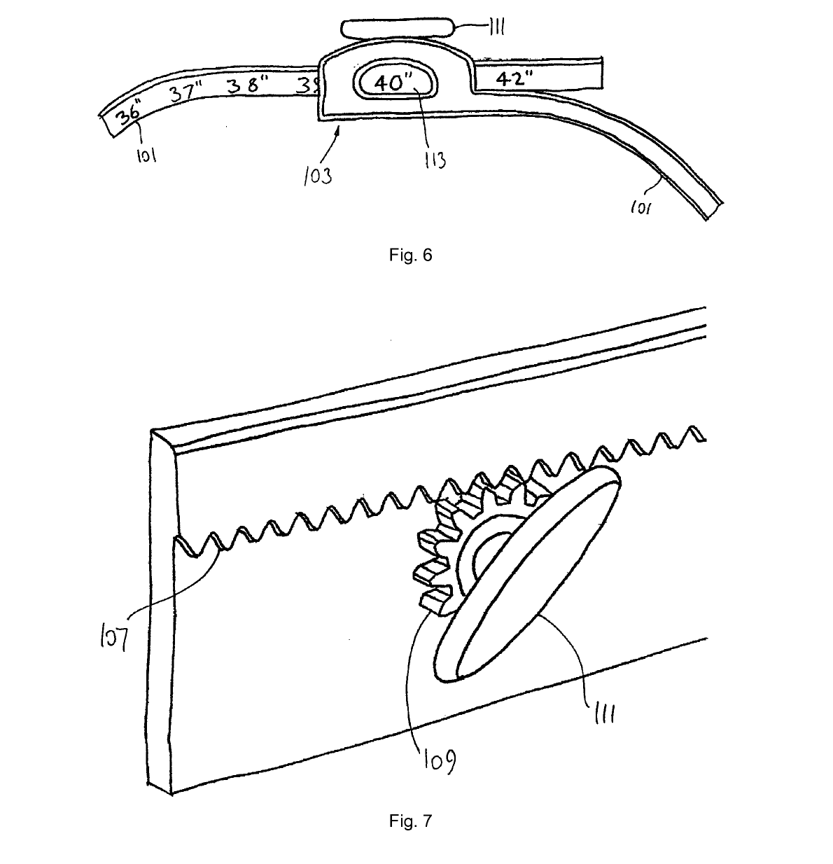

FIGS. 5-7 illustrate another part of the embodiment of the present invention shown in FIGS. 1-4;

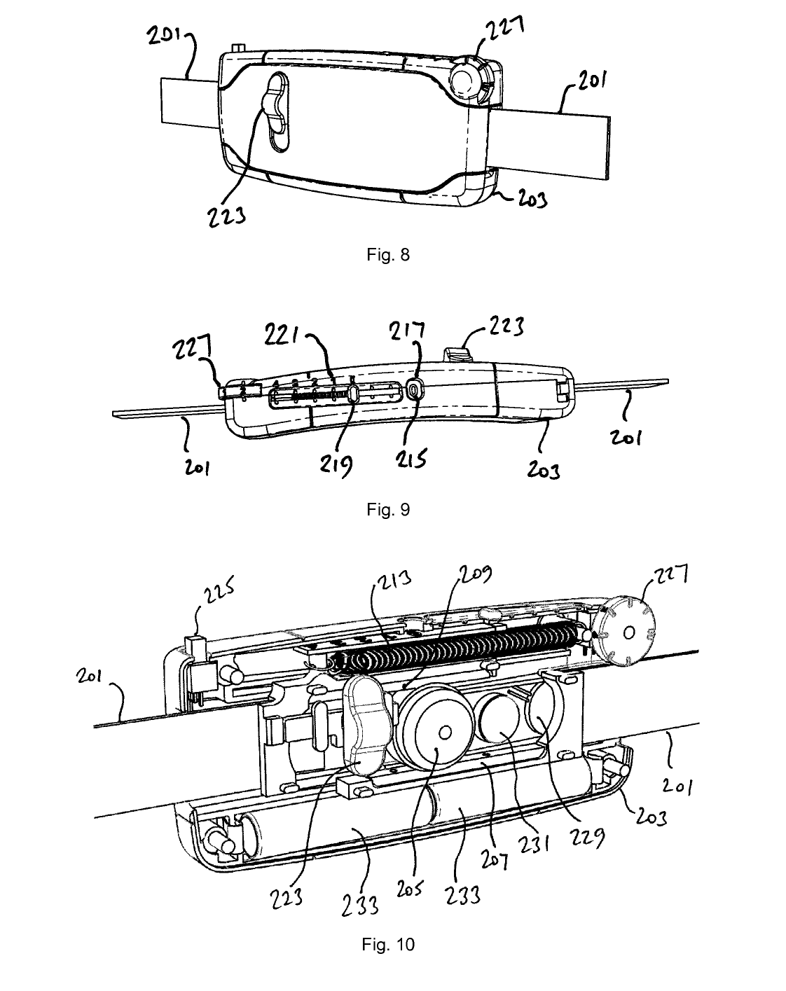

FIGS. 8-11 illustrate another embodiment of a belt according to the present invention for training abdominal muscles;

FIG. 12 is a front view of a further embodiment of a belt according to the present invention; and

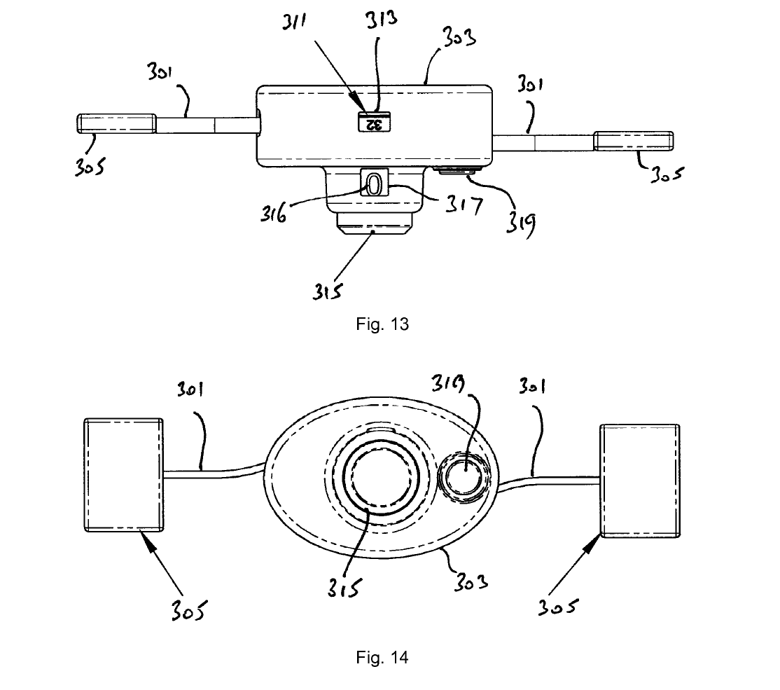

FIGS. 13 to 15 illustrate a further embodiment of a belt according to the present invention.

The belt shown in FIGS. 1 to 7 employs a mechanical system only and does not incorporate any electrical or electronic components, but it may be modified to incorporate electrical or electronic components as will be explained hereinafter. The belt comprises a strap 101 of substantially inelastic material for passing around the user's waist and securing with an adjustable clasp mechanism 103 which is positioned in use at the user's side or back. The belt includes a housing 105 which in use is positioned at the front of the user.

The adjustable clasp mechanism 103 incorporates a ratchet mechanism in the form of a row of teeth 107 formed in an edge region of the strap 101 and a rotatable gear 109 engaging with the teeth 107 and to which is mounted an external handle 111. The ratchet mechanism is tightened once the strap has been passed around the user's waist by rotating the handle 111, the amount of rotation of the handle being indicated in a window 113 provided in the clasp mechanism as the waist girth of the user (the girth displayed decreasing as the handle is rotated). The belt can be released by depressing the handle 111 to disengage the ratchet mechanism, although other release mechanisms can be provided (such as, for example, pulling the handle to disengage the ratchet mechanism).

The housing 105 contains a rack and pinion mechanism in the form of two rows of teeth 115, 117 with a rotatable gear wheel 119 acting between the upper and lower rows of teeth. One row of teeth is secured directly or indirectly to each end of the strap 101 so that any increase or decrease in the user's girth causes relative movement of the two rows of teeth and corresponding rotation of the gear wheel 119. The rack and pinion mechanism incorporates a spring tensioning means, for example in the form of a torsion spring 121 provided around a shaft of the gear wheel 119 which biases the strap so as to tighten around the user's waist (that is to reduce the girth of the belt). A scale is provided on one of the components 123 carrying one of the sets of teeth so as to indicate the amount of change in the user's girth during exercise. The scale is visible through one or more windows 125 (two windows being shown in the figures) and is conveniently a scale of 0 to 10. In use, when the belt is first put on, the user stops tightening the belt using the ratchet mechanism once the scale in the window 125 reads zero. At this time the user's waist girth is shown in the window 113 of the adjustable clasp 103.

A further scale 127 is provided on the housing 105 to indicate the maximum contraction during use of the belt. This is effected by providing a sliding indicator 129 on the component 123 which in use abuts against the end of a slot 131 in the housing 105 to move the sliding indicator along the component 123 in response to contraction of the user's transversus abdominis muscles. The sliding indicator 129 needs to be reset to zero before exercise commences.

Once the belt has been set up the user can begin to exercise by activating the transversus abdominis muscles. This in turn causes the user's girth to decrease and subsequently increase upon relaxation. As the user's waist girth decreases, the torsion spring causes the gear wheel 119 to rotate as the belt responds to the decrease in girth. The reduction in girth at any time is shown in the window 125 on a scale of 0 to 10 (that is the instantaneous degree of contraction of the user's abdominal muscles) while any change in the maximum decrease in girth is recorded by the sliding indicator 129 on the scale 127, again on a scale of 0 to 10.

Feedback to the user while exercising is generally by way of the window 125 and the sliding indicator 129 and provides a continuous and progressive feedback on the amount of contraction of the user's abdominal muscles which enables the user not only to learn how to activate his transversus abdominis muscles correctly, but also allows the user to be coached by a personal trainer/healthcare professional, if such coaching is necessary. However, an audible feedback may be provided in addition to the visual feedback from the window 125. That is means may be provided to generate an audible clicking sound when particular points are reached on the scale. An audible feedback allows the belt to be worn underneath clothing, when the visual feedback is not available.

Once the user has finished exercising, the handle 111 is operated to release the ratchet mechanism and to allow the belt to be removed.

The belt shown in FIGS. 1 to 7 may be modified for example, to provide or incorporate electronic, rather than mechanical, feedback. Thus, the audible feedback may be created electronically and/or tactile (e.g., vibratory) feedback bay be provided. Additionally, or alternatively, position sensing which gives rise to determining the change in the user's girth may be effected by employing an electrical transducer to convert positions into electrical signals rather than relying on mechanical components. Electrical signals allow further processing possibilities than mechanical components.

For example, the belt may be provided with a mode button which switches between a measurement mode and a training mode.

In the measurement mode the belt may provide a feedback signal to the user in response to a change in girth. The user's minimum or “personal best” girth may also be recorded in memory together with the time and date of the measurement, for subsequent recall by the user. The “personal best” may serve to record the progress of training with time and to determine the required change in girth to activate the feedback mechanisms during training. In addition, the memory may maintain a record of minimum girth with time so as to provide, for example, an indication of girth reduction with time due to increased muscle tone achieved through strengthening exercises undertaken using the belt, or loss of fat, such as through an associated diet and other exercise plan. Measurement mode may also allow the intensity of training (high, medium or low) to be selected.

In the training mode, the user may be required to achieve and/or maintain a predetermined percentage of their “personal best” (the percentage can be set by the user (high, medium or low)) in order to activate the feedback mechanism(s). The belt may have stored in memory one or more training routines for the user, which may be selected by the user or may be formulated specifically for the user. Thus, the belt may guide the user through a predetermined training programme defining all aspects of the user's training, including time of day, contraction magnitude, duration and number of abdominal muscle contractions. The belt may also store in its memory the number, duration and percentage of “personal best” abdominal muscle contractions achieved during the training session for comparison between the prescribed and achieved training. Both the ECG and EMG sets of data may be recorded during training for determining the intensity of the training (ECG) and the magnitude of the abdominal muscle activation (EMG). The time and date of each training session may also be stored in memory. The stored data may either be displayed on a display or may be exported to an external device in known manner.

As noted above, the level of the feedback signal may be determined by the magnitude of the user's abdominal girth. For example, in the case of vibration, a vibratory signal may be activated when the user achieves a threshold (low, medium or high) percentage of the user's personal best girth determined by the user. When the measured girth is less than the threshold, the intensity or amplitude of vibration may increase in proportion to the extent by which the threshold is exceeded, while as the measured girth increases towards the threshold the intensity or amplitude may decrease. Thus, the feedback may vary in response to fluctuations in the user's abdominal girth once the threshold has been achieved. In this way, the user can ensure that the target threshold is maintained so that maximal training benefits can be achieved.

A separate feedback signal may be activated, the particular signal being selected by the user, when the user maintains the threshold for a predetermined time and/or when the user completes a predetermined number of contractions achieving the threshold. That is, when the user completes the prescribed training.

The inelastic construction of the belt accommodates a predetermined maximum change in girth from the relaxed to the personal best contracted position. It has therefore been found that it is advantageous to base the threshold on the personal best girth measurement of the user, the threshold corresponding to a girth that is greater than the personal best girth measurement by a predetermined percentage.

In use of such a modified belt, as explained above the belt is initially worn under slight tension with the user standing in an upright posture, sitting, or lying on their back, and with the abdominal muscles contracted. A button may be pressed to initialise electronic circuitry once the correct personal best girth has been established, as indicated by the initial tension and/or belt length. After this set-up has been completed, the abdominal muscles can be relaxed and the user is ready to enter the training mode.

The first time the user wears such a modified belt, and from time-to-time thereafter, the user should enter the measurement mode, establish a “personal best” reduction in girth. Thereafter, the user can enter the training mode and train the abdominal muscles.

In order for intra-abdominal pressure and the associated training stimulus to be maximised, it has been found that it is important that all the abdominal muscles are contracted; if one or more of the muscles is relaxed, then some of the internal pressure generated by the contraction of the remaining abdominal muscles is dissipated. The training regime can also incorporate a specific breathing regimen which (i) ensures the maintenance of the intra-abdominal pressure, and (ii) increases the intra-abdominal pressure still further through the downward movement of the diaphragm (referred to as “maximal inhalation”) whilst maintaining the abdominal muscles contracted. To this end, the user contracts his or her abdominal muscles to achieve a predetermined reduction in abdominal girth which is indicated, where provided for, by a signal from the feedback mechanisms of the belt. The user then inhales deeply using the diaphragm while maintaining the abdominal girth, which intensifies the training stimulus for all the abdominal muscles by increasing the intra-abdominal pressure still further. The abdominal muscles are therefore trained by harnessing a training stimulus which is generated by the body itself. Failure to maintain contraction of the abdominal muscles, particularly the transversus abdominis and rectus abdominis, results in an increase in abdominal girth which is conveyed to the user by the feedback mechanisms of the belt. The user can therefore respond to the loss of training intensity by contracting the abdominal muscles to reduce the girth once again. Without the belt, the user would not be aware of the loss of training intensity, and the training would therefore have been relatively ineffective.

Further, a functional training system can be produced by combining the abdominal contraction and breathing exercises with specific functional body movements. The specific functional body movements are ideally such that they require functional activation of both the abdominal muscles and other deep core muscles. Such exercises might include, but are not limited to exercises that are currently used to train the abdominal muscles (such as sit-ups and “crunches”), static core training exercises (such as “the plank” and derivatives thereof), and lunging and balancing exercises that require the co-ordinated contraction of core muscles to control the body's centre of gravity.

The belt may additionally or alternatively be used to test the endurance of the user's abdominal muscles. In this case, the user makes repeated maximal contractions of their abdominal muscles in time with signals, for example provided by the belt. The resulting changes in girth are monitored and, as fatigue ensues, these become smaller. An index of abdominal muscle endurance may be derived by analysing the decline in girth reduction with time.

Thus the present invention provides a means of measuring the magnitude of abdominal muscle contraction, as indicated by the change in abdominal girth and thus eliminating, or at least reducing, the need for a personal trainer or physiotherapist. The change in girth also indicates the magnitude of the training stimulus to the muscles and the corresponding strength of those muscles. Thus, different (for example, high, medium and low) intensities of training can be achieved by specifying different magnitudes of abdominal girth reduction. The present invention also provides a means of monitoring progress by measuring the maximal extent of girth reduction and providing quantitative feedback on both training activity (contraction strength) and progress through training (changes in strength with time). The present invention also provides a means for undertaking a predetermined abdominal muscle training session at a time that the user defines, which may be during other daily activities, such as whilst working at a desk, by means of a reminder feature. In such a case, the belt can be worn discretely under the user's clothing. The present invention additionally provides a means for enabling the user to learn to activate and contract his abdominal muscles in such a way to be able to train key muscles, such as the transversus abdominis, that are not trained by conventional abdominal training methods.

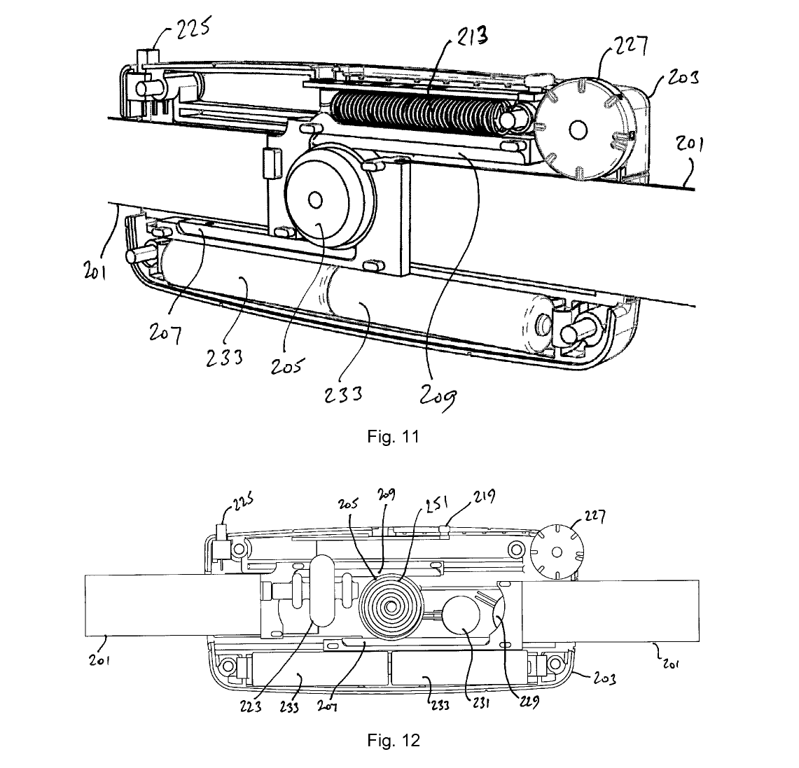

Another embodiment of a belt according to the present invention is shown in FIGS. 8 to 11 in which FIG. 8 is a front perspective view, FIG. 9 is a top view, FIG. 10 is a front perspective view corresponding to FIG. 8 with a cover removed and the belt in an expanded configuration, and FIG. 11 is a front perspective view corresponding to FIG. 10 with a lock removed and the belt in a contracted configuration.

The belt shown in FIGS. 8 to 11 comprises a strap 201 of substantially inelastic material around the user's waist, the belt including a housing 203 which in use is positioned at the front of the user. The housing 203 contains a rack and pinion assembly in the form of a rotatable pinion gear 205 engaging with a rack in the form of a lower row 207 and an upper row 209 of teeth. It should be noted, for clarity, the teeth are not shown in the figures, but are indicated diagrammatically. The lower row 207 of teeth of the rack is secured to one end of the strap 201 and is slidably mounted within the housing 203, while the upper row 209 of teeth is secured to the other end of the strap 201 and is also slidably mounted within the housing 203. The both the lower and upper rows 207, 209 of teeth are movable in conjunction with rotation of the pinion gear 205. The belt is mounted around the waist of the user by means of a releasable clasp (not shown) such as that described above in relation to FIGS. 1 to 7.

Any increase or decrease in the user's girth causes relative lateral movement between the lower and upper rows 207, 209 of teeth and the housing 203 which in turn causes rotation of the pinion gear 205. The upper row 209 of teeth is attached to one end of a coil spring 213 by way of a suitable post, the other end of the spring 213 being secured to the housing in order to provide a biasing force through the rack and pinion assembly to the strap 201 to bias the components to an initial configuration on which the belt is tightened around a user's waist.

A scale 215, for example in a range of from 0 to 4, is provided on the components with the upper row of teeth, which scale can be viewed through an opening 217 in an upper surface of the housing, and shows the amount of change in the user's girth during exercise. In use, when the belt is first put on, the user stops tightening the belt when the scale in the opening reads zero. A further scale 221 is provided on the housing to indicate the maximum contraction during use of the belt. This is effected by providing a sliding indicator 219 which is initially set to zero before exercise commences and, in use, slides relative to the housing in response to contraction of the user's transversus abdominis muscles. It will be noted that the zero setting of the scale 221 is not at the end of the potential range of movement of the indicator 219, which avoids the risk of over-tightening the belt. For example, the zero setting may be about one-third from the end of the potential range of movement.

A lock 223 is provided for the upper row 209 of teeth so as to prevent the rack and pinion mechanism operating. Upward movement of the lock results in engagement, while downward movement results in disengagement. The lock is designed such that it cannot be engaged unless the belt is extended to the zero point (that is, it cannot be locked in a contracted configuration) and allows the user more readily to put the belt on.

In use of the belt, the user first puts the belt around his waist, adjusts it to the girth of the waist and sets the maximum contraction indicator 219 to zero. Activation of the transversus abdominis muscles causes the user's girth to decrease and subsequently to increase upon relaxation. As the user's waist girth decreases, the spring 213 causes the ends of the belt to move together and to cause the pinion gear 205 to rotate as the lower 207 and upper 209 rows of teeth move relative to each other. The reduction in girth at any time is shown on the scale 215 through the opening 217 on a scale of 0 to 4 (that is, the instantaneous degree of contraction of the user's abdominal muscles), while any change in the maximum decrease in girth is recorded on the sliding indicator 219 relative to the scale 221, again on a scale of 0 to 4.

Feedback to the user while exercising is generally by way of the scale 215 and the sliding indicator 219 and provides a continuous and progressive feedback on the amount of contraction of the user's abdominal muscles which enables the user not only to learn how to activate his transversus abdominis muscles correctly, but also allows the user to be coached by a personal trainer/healthcare professional, if such coaching is necessary. However, an audible feedback may be provided in addition to the visual feedback from the scale 215. That is, means may be provided to generate an audible clicking sound when particular points are reached on the scale. An audible feedback allows the belt to be worn underneath clothing, when the visual feedback is not available.

The belt may be operated solely mechanically, but an electronic exercise guidance system is also provided. In such a case, an on/off button 225 is provided on the housing and a thumbwheel 227, which incorporates a rotary potentiometer, is provided adjacent to the indicator 219 and is adjusted by the user to the same value as the scale 221. The guidance system also includes a vibratory device 229, such as a vibration motor, and/or an audible device 231, such as a buzzer, together with one or more batteries 233. The electronic exercise guidance system also includes a rotary potentiometer forming part of the pinion gear 205, rotation of the pinion gear, and therefore of the potentiometer forming part of the gear, corresponding to the degree of contraction of the belt.

The thumbwheel 227 may incorporate a rotary encoder as an alternative to a rotary potentiometer. A rotary encoder has the advantage that it can rotate continuously. In such a case, an additional pushbutton may be provided. In use of such a modified belt, the user will initially place the belt around his waist and set the initial tension to be close to the zero point on the scale. The pushbutton is then pressed and the electronic exercise guidance system determines the actual position on the scale. The user then performs a maximum contraction and presses the button again once this has been achieved. This position is also determined by the electronic exercise guidance system and a target is established at a predetermined percentage, such as 70 percent, of the difference between the maximum contraction position and the starting position, that is the base waist girth of the user. Alternatively, the maximum contraction position may be established a predetermined time, such as 10 seconds, after the button is first pressed, allowing the user sufficient time to adopt a maximum contraction position. A vibratory device 229 and/or an audible device 231 may be used to indicate to the user when the predetermined time has expired. The belt may then be used with feedback being given as to the extent of the user's abdominal muscle contractions, for example as explained above in relation to the training mode.

The user may then fine-tune their exercise program by turning the thumbwheel, with the electronic exercise guidance system adjusting the threshold accordingly.

As an alternative to a rotary encoder and pushbutton, two pushbuttons may be provided, one pushbutton being used to increase the threshold and the other pushbutton being used to decrease the threshold.

The rotary potentiometer or rotary encoder incorporated into the thumbwheel 227 or the two pushbuttons allow the user to re-set the target point without having to re-tension the belt. This is particularly convenient where the user changes stance during the exercise program because it has been found that the user's resting waist girth, and therefore the target point, changes depending on whether the user is standing, lying or adopting an alternative posture.

If desired, the vibratory device 229 and/or the audible device 231 may emit a different signal to indicate that the user has expanded his waist beyond the zero point, such as might occur when undertaking a forward flexion movement in the lying position.

Although FIGS. 8-11 show upper and lower rows 207 of teeth forming part of the rack and pinion assembly, it is possible for one end of the strap to be secured directly to the housing and for one of the rows of teeth, in this case the lower row 207, to be omitted.

FIG. 12 shows that the coil spring 213 can be replaced by a spiral constant force spring 251 mounted about a pivot axis of the pinion gear 205 and positioned, for example, within the pinion gear.

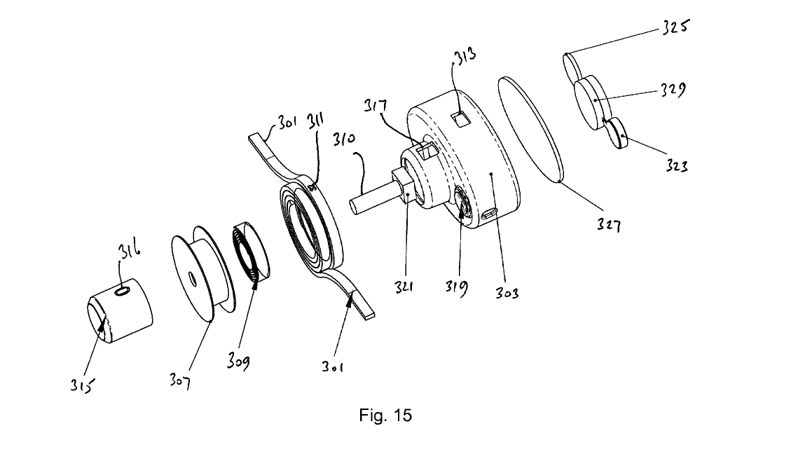

A further embodiment of a belt according to the present invention is shown in FIGS. 13 to 15 in which FIG. 13 is a top view, FIG. 14 is a front view and FIG. 15 is an exploded perspective view.

The belt shown in FIGS. 13 to 15 comprises a strap 301 in the form of a flat cable of substantially inelastic material to be placed around a user's waist, the strap portions extending from openings provided in a housing 303 which in use is positioned at the front of the user. The two strap portions are secured together by means of a buckle 305. The housing contains a spool 307, within which is mounted a spiral constant force spring 309, the spool being mounted to rotate with a spindle 310. The two ends of the strap 301 are coiled around the spool such that the spool rotates in a first direction (anticlockwise as illustrated) when belt material is pulled off the spool and in a second direction (clockwise as illustrated) when belt material is withdrawn into the housing and onto the spool under the biasing effect of the helical spring 309. One end of the strap has markings 311, or alternatively mouldings, (for example in inches) providing a scale to indicate the girth of the user's waist, which markings can be seen by the user through an opening 313. A rotatable indicator 315 which is provided with a scale 316 on its circumferential surface (only the value zero being illustrated) is a friction fit on the spindle 310, the scale being visible to the user through a further opening 317 provided in the housing 303. In use of the belt, the scale 316 is set to zero as a starting point by releasing and re-applying the indicator 315. Activation of the user's transverse abdominal muscles causes the user's waist girth to decrease and subsequently to increase upon relaxation. As the user's waist girth decreases, the spring causes the strap 301 to be drawn onto the spool 307 and to cause the spindle 310, and therefore the indicator 315, to rotate and the reduction in girth at any time is indicated on the scale 316 (that is, the instantaneous degree of contraction of the user's abdominal muscles). Feedback to the user while exercising is generally by way of the scale 316 and provides a continuous and progressive feedback on the amount of contraction of the user's abdominal muscles which enables the user not only to learn how to activate his transversus abdominis muscles correctly, but also allows the user to be coached by a personal trainer/healthcare professional, if such coaching is necessary. However, an audible feedback may be provided in addition to the visible feedback from the scale. That is, means may be provided to generate an audible clicking sound when particular points are reached as the spindle 310 rotates. An audible feedback allows the belt to be worn underneath clothing, when visual feedback is not available.

The belt has an electronic exercise guidance system which is optional. A control button 319 is provided on the housing 303 and the threshold is adjusted by pressing the button. The guidance system also includes a vibratory device 323, such as a vibration motor, and/or an audible device 325, such as a buzzer, together with a circuit board 327 which carries electronic components for the electronic exercise guidance system and one or more batteries 329. In use, the user will initially place the belt about his waist and adjust the girth and the mechanical zero setting. The pushbutton 319 is then pressed and the electronic exercise guidance system determines a starting point. The user then performs a maximum contraction and presses the button again once this has been achieved. This position is also determined by the electronic exercise guidance system and a target is established at, say, 70 percent of the difference between the maximum contraction position and the starting position. Alternatively, the maximum contraction position may be established a predetermined time, such as 10 seconds, after the button is first pressed, allowing the user sufficient time to adopt a maximum contraction position. The vibratory device 323 and/or the audible device 325 may be used to indicate to the user when the predetermined time has expired.

The belt may then be used with feedback being given as to the extent of the user's abdominal muscle contractions, for example as explained above in relation to the training mode.

1. A belt for training abdominal muscles comprising: means (103; 305) for determining a base girth of a user; means (115, 117, 119; 205, 207, 209; 301, 307, 310, 321) for determining changes in girth of the user as a result of contraction and relaxation of the user's abdominal muscles; means (125; 215; 316) for providing feedback to the user as to the extent of contraction of the user's abdominal muscles, the feedback means comprising means (125; 215; 316) for displaying a continuous, progressive indication of the degree of contraction of the user's abdominal muscles.

2. A belt as claimed in claim 1, wherein the feedback means (125; 215; 316) includes a scale which indicates the degree of contraction of the user's abdominal muscles.

3. A belt as claimed in claim 1, wherein the feedback means (127,129; 219, 221) includes means for indicating a maximal contraction of the user's abdominal muscles.

4. A belt as claimed in claim 3, wherein the means for indicating maximal contraction comprises a scale (127; 221).

5. A belt as claimed in claim 1, wherein a belt member (101; 201; 301) for extending around a waist of a user is substantially inelastic.

6. A belt as claimed in claim 5, wherein the belt member (101; 201; 301) is connected at each end thereof to the means (115, 117, 119; 205, 207, 209; 301, 307, 310, 321) for determining changes in the girth of the user, which means includes means for adjusting the length of the belt member at each end thereof.

7. A belt as claimed in claim 6, wherein the means for adjusting the length of the belt member (101; 201; 301) includes a rotatable member (119; 205; 307).

8. A belt as claimed in claim 6, wherein the means for adjusting the length of the belt member (101; 201; 301) includes biasing means (121; 213; 251; 309) which tends to tighten the belt member around a user's waist.

9. A belt as claimed in claim 6, wherein the means for adjusting the length of the belt member (101; 201) comprises a rack and pinion assembly (115,117,119; 205, 207, 209), a rack portion (115,117; 207,209) being attached to each end of the belt member and a rotatable pinion (119; 205) arranged in engagement with the two rack portions.

10. A belt as claimed in claim 6, wherein the means for adjusting the length of the belt member (301) comprises a spool (307) upon which two strips of the belt member are wound.

11. A belt as claimed in claim 1 and including means for determining when the girth of the user attains a predetermined threshold relative to the difference between the base girth and a girth corresponding to the user's maximum contraction of the abdominal muscles.

12. A belt as claimed in claim 11 and including means (229, 231; 323, 325) for varying the feedback to the user as the user's girth varies beyond the predetermined threshold.

13. A belt as claimed in claim 1, wherein the means for providing, feedback is selected from audio feed-back means (231; 325), visual feedback means (125; 215; 316), and tactile feedback means (229; 323), and combinations thereof.

14. A belt as claimed in claim 1 and including means for recording at least one of the user's minimum girth and the user's base girth.

15. A belt as claimed in claim 14 and including means for recording at least one of the girths over time.

16. A belt as claimed in claim 1 and including means for recording at least one of the duration of training and the combination of duration and number of reductions in girth.

17. A belt as claimed in claim 1 and including means for providing the user with feedback when the user maintains the predetermined threshold for at least one of a predetermined period of time and a predetermined number of times.

18. A belt as claimed in claim 1 and including a means of reminding the user that it is time to take a scheduled training session.

19. A training method employing a belt as claimed in claim 1, comprising the steps of: placing the belt around the waist of a user and determining a base girth of the user; and contracting and relaxing the user's abdominal muscles so as to provide feedback to the user as to the extent of contraction of the user's abdominal muscles; and noting a continuous, progressive indication of the degree of contraction of the user's abdominal muscles.

20. A method according to claim 19 and including the further step of the user inhaling while maintaining a reduction in abdominal girth.

21. A method according to claim 19 and including the step of testing the endurance of the user's abdominal muscles.

22. A method according to claim 21, wherein in the endurance testing step the user makes repeated maximal contractions of the abdominal muscles in time with signals provided by the belt.

23. A method according to claim 19 and including the step of deriving an index of abdominal muscle endurance by analysing the decline in girth reduction with time.
